(19) United States
(12) Patent Application Publication
(10) Pub. No.: US 2006/0293388 A1
(43) Pub. Date: Dec. 28, 2006

(54) Pomolic acid, its isomers, derivatives and their uses, pharmaceutical composition, method to prepare the pharmaceutical composition and method for treating multidrug resistant tumours

(75) Inventors: Cerli Rocha Gattass, Rio de Janeiro (BR); Vivian Mary Dodd Rumjanek, Rio de Janeiro (BR)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: UNIVERSIDADE FEDERAL DO RIO DE JANEIRO, Rio de Janeiro, RJ (BR)

(21) Appl. No.: 10/530,333

(22) PCT Filed: Oct. 2, 2003
(86) PCT No.: PCT/BR03/00144

§ 371 Date: Aug. 18, 2005

(51) Int. Cl. C07C 61/29; A61K 31/20

(52) U.S. Cl. 514/559; 562/403

(57) ABSTRACT

The present invention describes the use of pomolic acid, its derivatives and pharmaceutical preparations derived from them as anti-neoplasic agents in the treatment of multidrug resistant tumors. In relation to other drugs that present anti-MDR activity, these substances do not require the concomitant use of reversers to exert their anti-MDR activities.

### I. FIELD OF THE INVENTION

The present invention is related to a substance for the treatment of multidrug resistant tumors, to a composition containing the substance, to the method of preparing this composition, to the use of this substance for the preparation of anti-cancer medicaments, to the use of this substance for the treatment of cancer, as well as the method of treatment of patients with multidrug resistant tumors.

The present invention is specifically related to the identification of pomolic acid, its isomers and derivatives as anti-neoplastic drugs, to be used in the treatment of patients suffering from tumors intrinsically multidrug resistant or tumors that acquired this resistance as a result of chemotherapy treatment.

### II. INVENTION BACKGROUND

The treatment of human cancer is a branch of medicine that presents still unsolved challenges. Despite the fact that in medicine treatments presenting a greater efficacy and capable of producing the cure of various types of cancer have been developed, these treatments lead many times to undesirable side effects in the patients.

As a result of these problems, new techniques need to be created to solve the existing problems in the present methods. For example, prostate cancer which is the most common cancer in man and responsible for most deaths in this gender, can be treated surgically, using medicaments (chemotherapy/hormones), by radiotherapy or using a combination of these treatments, depending on the stage of the disease.

Among the many existing therapies, chemotherapy is the most promising one for the treatment of the various types of cancer. However, despite the high efficacy of some drugs, the majority of them present serious side effects which preclude their use for prolonged periods of time or repeatedly. For this reason, patents U.S. Pat. No. 5,558,866 and U.S. Pat. No. 5,876,728 propose the use of natural substances, obtained from various plant species, such as plants of the Pittosporaceae family, as the source of new, less toxic chemotherapeutic drugs. That is, drugs with a greater selectivity for the treatment of the tumor resulting in a lesser aggression to the metabolism of the patients' normal cells.

Nowadays, there are several drugs that are used against cancer. Many of them are used alone whereas others show a greater efficacy when used in combination with other drugs or with other therapies.

The U.S. Pat. No. 5,602,184 shows the use of some terpenes, chemotherapeutic agent that presents little or even no toxicity, for the treatment of metastatic cancer. This same reference states that treatment with terpenes, selected among acyclic and cyclic monoterpenes, acyclic sesquiterpenes and/or acyclic diterpenes, increases the susceptibility of treated cancer cells to radiotherapy, demonstrating that the combined use of therapeutic methods, despite elevating treatment costs, may produce more promising results for cancer cure.

In more recent studies, the U.S. Pat. No. 5,587,402 shows that treatment with limonene, a monoterpene present in the oil of orange skin, has an effect against many types of cancer, such as breast cancer, stomach and lung cancer. However, despite being considered as a non-toxic chemotherapeutic agent for humans in some doses, limonene presents some undesirable side effects, particularly when used in high dosages and short time intervals. As a result of this, their inventors presented another method to produce inhibition or regression of leukaemias, without using limonene, but using instead perillyl alcohol.

In spite of the existence of a great amount of chemotherapeutic agents being produced in the constant struggle against cancer, the efficacy of chemotherapy has been prejudiced by the specific resistance of some tumors to certain chemotherapeutic agents or by the presence in the patients of multidrug resistance, a phenomenon that may be inherent to the patient or acquired as a result of the treatment.

The phenomenon of multidrug resistance (MDR) involves the cross resistance among a number of non-related chemotherapeutic drugs that diverge in respect to their chemical structure, mode of action and cellular target. This group of factors makes MDR one of the main reasons for the chemotherapy failure seen in many tumors.

The MDR phenomenon is multifactorial resulting from defects in the regulation of the genes that control apoptosis, an increase in the process of cellular detoxification, alterations in the DNA repair system and activation or over expression of drug transporter proteins such as P glycoprotein (Pgp), the protein related to multiresistance (MRP) and the protein related to lung resistance (LRP).

Because in MDR, some of the drug transporter proteins function as efflux pumps, removing the chemotherapeutic agent from the cell, one of the strategies utilized nowadays for the treatment of tumors expressing MDR is the use of reversers of the pumps, associated to chemotherapy. The U.S. Pat. No. 5,541,232 presents a method for preventing the development of multidrug resistance (MDR) and to revert the existence of multidrug resistance in case it already exists, trough the prevention or correction of the defect in drug accumulation by resistant cells. More particularly this reference describes the administration of MASOPROCOL jointly with anti-neoplasic/cytotoxic drugs that induce the development of multidrug resistance in cells.

The patent EP 941737, despite not approaching the problem of multidrug resistance, presents some inducers of apoptosis in cancer cells. The main point of these products is to inhibit the drug efflux produced by Pgp, as the reduction of the intracellular concentration of the chemotherapeutic agent, is one of the main responsible for the inefficacy of some drugs which would lead to cellular apoptosis in the treatment of cancer. Despite the inhibition of this pump being mentioned in this reference as the cancer treatment per se, in the majority of times the use of efflux pump inhibitors in the treatment of tumors presenting multidrug resistance must be associated with a chemotherapeutic agent to produce the desired effect.

Other patents such as U.S. Pat. No. 5,916,566, do also present methods to inhibit the resistance mediated by glycoprotein P (Pgp) to pharmacological compounds by increasing their bio disposal. In this case, cited as a reference, essential oils are utilized to inhibit the activity of P450 or Pgp, which normally are the responsible for the elimination of these compounds.

Taking into account that the MDR phenomenon is one of the main causes of lack of success in tumor chemotherapy, and taking into account that chemotherapeutic agents capable of destroying these types of tumors, avoiding patient's death, are rare, the search for new anti-neoplastic drugs with anti-MDR activity, becomes imperative.

### III. SUMMARY OF THE INVENTION

One of the first embodiments of this invention consists in the identification of the activity of pomolic acid, its isomers and derivatives as an anti-neoplastic agent, more specifically, a substance with action against tumors that present multidrug resistance.

A second embodiment of this invention consists in a pharmaceutical composition containing pomolic acid.

A third embodiment of this invention is in the preparation of the pharmaceutical composition containing pomolic acid.

A fourth embodiment of this invention is related to a method to treat cancer presenting multidrug resistance, utilizing pomolic acid as a therapeutic agent.

A fifth embodiment of this invention is related to the use of pomolic acid for the preparation of medicaments for the treatment of cancer with multidrug resistance.

A sixth and last embodiment of this invention is related to the use of pomolic acid in the treatment of cancer with multidrug resistance.

### IV. BRIEF DESCRIPTION OF THE FIGURES

FIG. 1 describes the inhibition of proliferation of the leukaemic cell line K562 by pomolic acid. K562 cells were treated with 1, 10 and 100 μg/ml of pomolic acid for 48 hours. The values represent the mean±standard error of three independent experiments.

FIG. 2 describes the inhibition of proliferation produced by pomolic acid in the presence of vincristine, on the resistant leukaemia cell line Lucena 1. Lucena 1 cells were treated with 1, 10 and 100 μg/ml of pomolic acid for 48 hours. The values represent the mean±standard error of three independent experiments.

FIG. 3 describes the inhibition of proliferation produced by pomolic acid in the absence of vincristine, on the resistant leukaemia cell line Lucena 1. Lucena 1 cells were treated with 1, 10 and 100 μg/ml of pomolic acid for 48 hours. The values represent the mean±standard error of three independent experiments.

FIG. 4 shows the comparison between FIGS. 2 and 3 in the same graph, comparing the inhibition of proliferation produced by pomolic acid, in the presence or absence of vincristine, on the resistant leukaemia cell line Lucena 1. Lucena 1 cells were treated with 1, 10 and 100 μg/ml of pomolic acid for 48 hours. The values represent the mean±standard error of three independent experiments.

FIG. 5 shows the cytotoxic activity of pomolic acid over the cell line HL-60, another sensitive lineage. HL-60 cells were treated with 1, 5, 10, 25, 50 and 100 μg/ml of pomolic acid for 48 hours. The values represent the mean±standard error of three independent experiments.

FIG. 6 shows the cytotoxic activity of pomolic acid over the cell line Caco-2. Caco-2 cells were treated with 1, 5, 10, 25, 50 and 100 μg/ml of pomolic acid for 48 hours. The values represent the mean±standard error of three independent experiments.

FIG. 7 shows the cytotoxic activity of pomolic acid over the Ma104 cell line. Ma104 cells were treated with 1, 5, 10, 25, 50 and 100 μg/ml of pomolic acid for 48 hours. The values represent the mean±standard error of three independent experiments.

FIG. 8 represents the effect of pomolic acid (50 μg/ml) over different cell concentrations of the cell lines Caco-2 and Ma104. Results express the percent inhibition of viability after 48 hours treatment using the acid.

### V. DETAILED DESCRIPTION OF THE INVENTION

Patients with any kind of tumor, such as those of the ovaries, breast, lung, colon and many others, may eventually develop MDR.

The MDR phenomenon has been associated to the over expression of MDR genes that code for transporter proteins expressed in the plasma membrane, such as the glycoprotein P (MDR-1 gene) and the protein of multidrug resistance—MRP (MRP-1 gene). The expression of these genes is associated with a reduced cellular concentration of drugs, resulting from an energy-dependent active efflux mechanism of chemical compounds, as seen with Pgp, or of a mechanism dependent of the conjugation of these compounds with glutathione, as seen with MRP. Other genes also associated to the MDR phenomenon, code for proteins expressed in intracytoplasmatic vesicles, such as lung resistance protein—LRP, that is implicated in nuclear-cytoplasmic trafficking and in this way confers resistance to DNA-binding drugs. The great importance of these proteins to the phenomenon of multidrug resistance in tumor cells makes compounds, capable of modulating their activity, in powerful drugs for the treatment of cancer with this type of resistance.

The multiple resistance induced by the over expression of Pgp at the plasma membrane may be reversed in vivo and in vitro by a variety of hydrophobic, structurally and functionally unrelated substances, known as MDR reversers, modulatory agents or chemosensitizers. These substances block the efflux pump allowing the intracellular accumulation of the chemotherapeutic agent. The first substances utilized as reversers were the calcium channel inhibitor verapamil, the immunosuppressor cyclosporin A and a group of substances with known activity in other biological systems and totally unrelated among themselves such as phenotiazines, antimalarials, antibiotics, etc.

Second- and third-generation modulators are already being produced and being evaluated regarding their reversal capacity. Some of these agents, are now being used in patients in combination with chemotherapics with variable toxicity profiles. The toxicity observed derives from the pharmacological action of the drug being used as a reverser or from the fact that Pgp is also expressed in some normal tissues where the physiological role of this protein is unknown. Therefore, new chemotherapeutic agents that are not substrates for MDR transporters or that could function as reversers have a very high pharmaceutical interest.

However, the available pump reversers, do not show efficacy for all kinds of MDR tumors. This occurs because technically each type of cancer (leukaemia, breast, colon, lung and others) may respond in a different way to the chemotherapic.

Therefore, some patients bearing the same types of cancer, may eventually express the MDR (multidrug resistance) phenotype either intrinsically or as a result of the patient treatment with the chemotherapic. As the MDR phenomenon involves cross-resistance to structurally unrelated drugs that differ in their structure, mode of action and cellular target, treatment of these patients with the know chemotherapeutic drugs are totally without efficacy. In this case the patient will die unless a drug is identified, capable of circumventing or inhibiting the mechanisms activated by the MDR phenotype and in this way kill the cell.

In this way, the identification of a new drug with anti-MDR properties, especially if this drug is not a substrate for the transporter proteins, will always be of great clinical relevance, as it may represent the only alternative of cure for patients that showed resistance to the chemotherapeutic drugs available.

The present invention represents the offer of a new drug for the treatment of tumors expressing MDR and another alternative of treatment for patients bearing this type of tumor without the need of other drugs or additional therapies.

The fact that the drug has a direct effect over the tumor, avoids the need for the association of reversing substances, decreasing the risk of possible secondary effects that they may cause to the patient and being able to reduce treatment costs.

The substance of the present invention is a terpene, more specifically a triterpene, obtained from natural sources, such as plants, or obtained by chemical synthesis.

The triterpenes belong to a big family of compounds known as cycloesqualenoids, derived from the secondary metabolism of plants. In the last years many biological activities have been attributed to compounds that belong to this class, including anti-fungal, anti-inflammatory, anti-HIV and more recently anti-tumor activities.

When studying this class of substance, through the screening of anti-tumor agents, we observed that a reasonable number of bioactive compounds could be identified with anti-proliferative and cytotoxic activity for tumor cell lines; such as Taxol, terpenes, Yamagishi et al. 1988; Kim et al. 1998, 2000, betulinic acid, a triterpenoid isolated from the bark of Physocarpus intermedius, has been characterized as a potent tumoricidal agent inducing apoptosis in tumors of neuroectodermic origin, Schmidt et al. 1997; Fulda et al., 1997, 1998, 2001 and acting over melanomas in vitro and in vivo, Kim et al., 2000; Pisha et al., 1995; Raisova et al., 2001; and oleanoic acid, another triterpene isolated from the same plant, inhibits the growth of tumor cell lines in vitro, Kim et al., 2000, the angiogenesis, Sohn et al., 1994, and the development of tumors induced by TPA, Tokuda et al., 1996. Most recent works established that, in the same way as betulinic acid, other triterpenes are also capable of inducing the process of programmed cell death in tumor cells, Konopleva et al., 2002.

However, contrary to what one may believe, despite the great number of studies already carried out, the present inventors have been capable of recognizing among the triterpenes, more specifically in pomolic acid and its derivatives, a biological activity never tested before.

Therefore, despite the cytotoxic activity of this triterpene in melanoma cell lines had been already described in the literature, Neto et al., 2000, data presented in this document demonstrate the cytotoxicity of pomolic acid on cells that express the MDR phenotype. This represents a great advance in the capacity to kill tumors that are resistant to conventional chemotherapies.

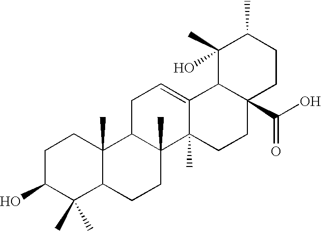

As mentioned above, pomolic acid is a molecule derived from plants and well know in the literature.

In this way, the identification of the active substance of this invention as being pomolic acid was easy, using for this studies of magnetic resonance already present in state of technique by Maillard M, Adwunmi C O, Hostettman K. A triterpene glycoside from the fruits of Tetraplura tetraplera. Phytochemistry 1992, 31(14), 1321-1323: Kakuno T, Shikawa K Y, Arihara S. Triterpenoid saponmins from Ilex crenata fruit. Phytochemistry 1992; 31(10, 3553-3557. In this way, we may assure that pomolic acid, according with the present invention, is the substance responsible for the cytoxicity on cancer cells presenting resistance to multiple drugs.

Therefore, this invention presents pomolic acid, its isomers and derivatives as a new substance with anti-MDR properties, capable of eliminating cancer cells and with low or no toxicity to the patients' normal cells.

### VI. EXAMPLES FOR ILLUSTRATION

With the objective of obtaining, testing and proving the efficacy of pomolic acid, its isomers and derivatives as a chemotherapeutic drug with anti-MDR activity, many studies were realized as shown below.

### Example 1

### Method for Obtaining Pomolic Acid

a. Summary of the methodology: Pomolic acid was obtained through the successive extraction of leaves with organic solvents, followed by the fractionation of the extract in silica gel and amberlite columns. The purity was characterized through the measurements of CG/MS, ¹H and ¹³C NMR, the point of fusion and optical rotation and its identification was performed comparing the physical and spectral data with that of the triterpenes already described in Maillard et al., 1992; Kakuno et al., 1992; Mahato et al., 1994.

b. Detailed methodology: Methanolic extracts of the dry and lyophilized leaves of Crysobalanus icaco L. were successively fractionated with hexane, CH₂Cl₂, AcOEt and BuOH. Pomolic acid was obtained from the CH₂Cl₂ fraction of the leaves of Chrysobalanus icaco L. through the fractionation in silica gel column followed by elution in a mixture of hexane/ethyl acetate (50%). This fraction was re-chromatographed in Amberlite ZAD-2 and the elution with methanol resulted in a white solid that presented the profile of a pure substance in thin layer chromatography. The purity of the compound was corroborated through the measurements of CG/MS, ¹H and ¹³C NMR, the point of fusion and optical rotation. The compound was identified as pomolic acid by comparison with the physical and spectral data of triterpenes already described in Maillard et al., 1992; Kakuno et al., 1992; Mahato et al., 1994.

### Example 2

### Evaluation of the Inhibition of Cellular Proliferation

As tumors grow spontaneously, one of most common methods to test the anti-tumor activity of a drug is to estimate the effect of the drug on the inhibition of cell proliferation. For these studies sensitive or multidrug resistant cell lines can be used.

The inhibition of cellular proliferation was measured through the incorporation of radioactive thimidine (3H-TdR). For this, 180 μl of the cell suspensions are delivered in wells of a 96 wells culture microtiter plate, 2×10⁴ cells/well, followed by incubation in a CO₂ incubator, at 37° C. for 24 h. After this period, 20 μl of RPMI (control) or the drug to be tested at the concentrations of 100 μg/ml, 50 μg/ml, 25 μg/ml, 10 μg/ml and 1 μg/ml, are added to each group of four wells. Some wells, to be used as controls, receive 20 μl of DMSO in the same concentrations carried by the drugs. Eight hours before the end of the culture, the plate received a pulse of radioactive thymidine (1 μCi/well) and radioactivity was measured in a β counter. The percentage of inhibition was calculated taking as a reference cell growth in the presence of DMSO.

These results are demonstrated in FIGS. 1 to 3, showing that pomolic acid inhibits both the proliferation of sensitive cell lines (K562) and multidrug resistant cell lines (Lucena), and that its effect is dose-dependent.

### Example 3

### Evaluation of Cytotoxicity

One of the characteristics wanted from a chemotherapeutic drug is its capacity of killing a tumor cell. This activity may be estimated by the quantification of the number of dead cells or the number of cells that remain viable after treatment. Measurements of DNA fragmentation indicate the mechanism (necrosis or apoptosis) through which the drug is acting.

The evaluation of the capacity of pomolic acid, its isomers and derivatives of killing tumor cells will be performed in vitro by the MTT method. The MTT method (3-4,5-dimethylthiazol-2yl)-2,5-diphenyltetrazolium bromide), that is based on the reduction of this compound to Formazam by the enzyme NADH dehydrogenase, Mosmann 1983, allows the quantification of the number of viable cells after treatment. For this, cells from the different cell lines will be processed as described above, distributed into plates and treated with the drug desired concentrations (1, 5, 10, 25, 50 or 100 μg/ml) or DMSO. After 48 h, 20 μl of MTT (5 mg/ml) will be added to each well and the plate will be incubated for a further 4 h in an incubator at 37° C. Following centrifugation, the formazan crystals will be dissolved with DMSO (200 μl/well) and the reading of the absorbance will be made in an ELISA reader at 570 nm wavelength. The percentage of inhibition of cellular viability will be calculated using as reference cells treated with DMSO.

The results presented in FIG. 4 show that the toxicity of pomolic acid grows as the dose increases. These results also showed the efficacy of the anti-tumor activity of pomolic acid over leukaemic cell lines other than K562 and Lucenal.

### Example 4

### Evaluation of Apoptosis Induction

A large number of chemotherapeutic drugs lead tumor cells to death through the induction of apoptosis. Measurements of DNA fragmentation indicate the mechanism (necrosis or apoptosis) through which the drug is acting.

The effect of pomolic acid over DNA fragmentation was studied through the analysis of the cell cycle by flow cytometry. For this 90 μl of cells (2.5×10⁵ cells/well) will be distributed in 96 well plates to which it will be added 10 μl of medium, DMSO or the drugs under test (10, 25, 50 and 100 μg/ml). After the defined period of time the cells will be harvested and spun down at 240 g for 5 min. The pellet will be resuspended in 300 μl of a HFS solution (HFS: 0.1% of Triton x-100, 0.1% of sodium citrate and 50 μg/ml of propidium iodide) and incubated at 4° C. for 1 h, in the absence of light. The samples will be read in a flow cytometer (Becton and Dikinson) using the FL2 channel.

Table 1 shows that treatment with pomolic acid produces DNA fragmentation in the cells. The K562 cells were treated with 10, 25, 50 and 100 μg/ml of pomolic acid for 18 h, lysed by HFS treatment (that contains propidium iodide) and DNA fragmentation was measured by FACS (flow cytometry). The values represent the mean±SD of three independent experiments.

TABLE 1
Percentage of DNA fragmentation in K562 cells
treated with pomolic acid.

| | Drug concentration (μg/ml) | | | |
|---|---|---|---|---|
| Treatment | 10 | 25 | 50 | 100 |
| *control | 5.08 ± 0.25 | 5.05 ± 0.27 | 4.91 ± 0.20 | 5.26 ± 1.09 |
| pomolic | 4.31 ± 1.21 | 13.72 ± 0.52 | 29.93 ± 2.74 | 74.88 ± 5.69 |

*Cells were treated with DMSO in the same concentrations carried by the drug dilution. Results represent the mean ± SD of 3 experiments.

DNA fragmentation described in table 1, demonstrates that the cytotoxic effect of pomolic acid is mediated by apoptosis induction.

### Example 5

### Estimation of the Reverser Activity of Pomolic Acid

A possible reverser activity of pomolic acid was evaluated by testing its effect over Lucena 1 cell line, that over expresses P glycoprotein (Pgp).

Table 2 demonstrates the resistance of Lucena 1 cells and the sensitivity of K562 cells to the chemotherapeutic agent Vincristine, a drug largely utilized in cancer treatment. Rumjanek et al. (2001) have shown the resistance of Lucena 1 to other chemotherapeutic agents characterizing it as a MDR cell line.

TABLE 2
Effect of vincristine (VCR) on the growth of K562
and Lucena 1. Results express the percentage of survival in
relation to the untreated control.

| Treatment | K562 (%) | Lucena (%) |
|---|---|---|
| Control | 100 | 100 |
| VCR 60 nM | 4.9 ± 3.1 | 97.8 ± 3.7 |
| VCR 30 nM | 10 ± 2.9 | 99.5 ± 6.5 |
| VCR 15 nM | 29.7 ± 7.7 | 97.4 ± 6.3 |
| VCR 7.5 nM | 70.1 ± 11.8 | 98.4 ± 1.9 |
| VCR 3.75 nM | 76 ± 7.8 | 106.6 ± 3.8 |

Table 3 shows that the use of modulators of the efflux pump, such as cyclosporin A (CSA) and verapamil (VP) do not affect Lucena's growth but are necessary to guarantee the efficacy of the chemotherapeutic agent vincristine (VRC) over this cell line. This result indicates that Lucena 1 resistance is mediated by Pgp.

TABLE 3
Modulatory effect of cyclosporin A (CSA) and
verapamil (VP) on the growth of K562 and Lucena 1 cells.
Results express the percentage of survival.

| Treatment | K562 (%) | Lucena (%) |
|---|---|---|
| Control | 100 | 100 |
| VCR 60 nM | 3.1 ± 1.5 | 105.3 ± 7.3 |
| CSA 160 nM | 101.8 ± 3.4 | 105.2 ± 5.3 |
| CSA/VCR | 1.8 ± 0.9 | 2.6 ± 1.4 |
| VP 5 μM | 106.3 ± 2.2 | 103.5 ± 3.0 |
| VP/VCR | 2.9 ± 0.7 | 3.6 ± 1.6 |

To test if pomolic acid acted as a Pgp reverser its effect on the growth of Lucena 1 was evaluated in the presence and absence of the chemotherapeutic agent vincristine. The experimental design was the same one as described in example 1.

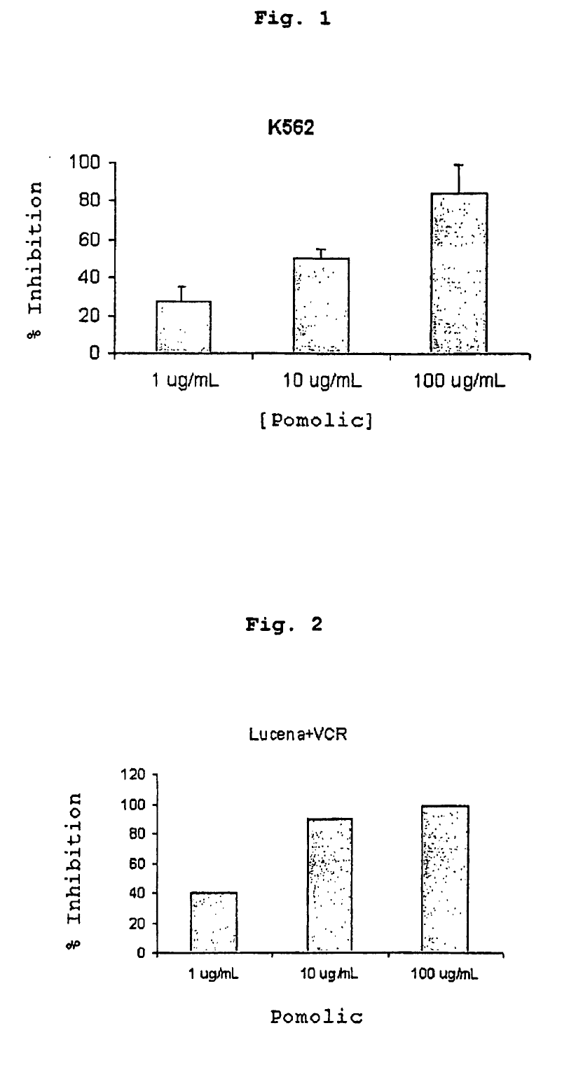

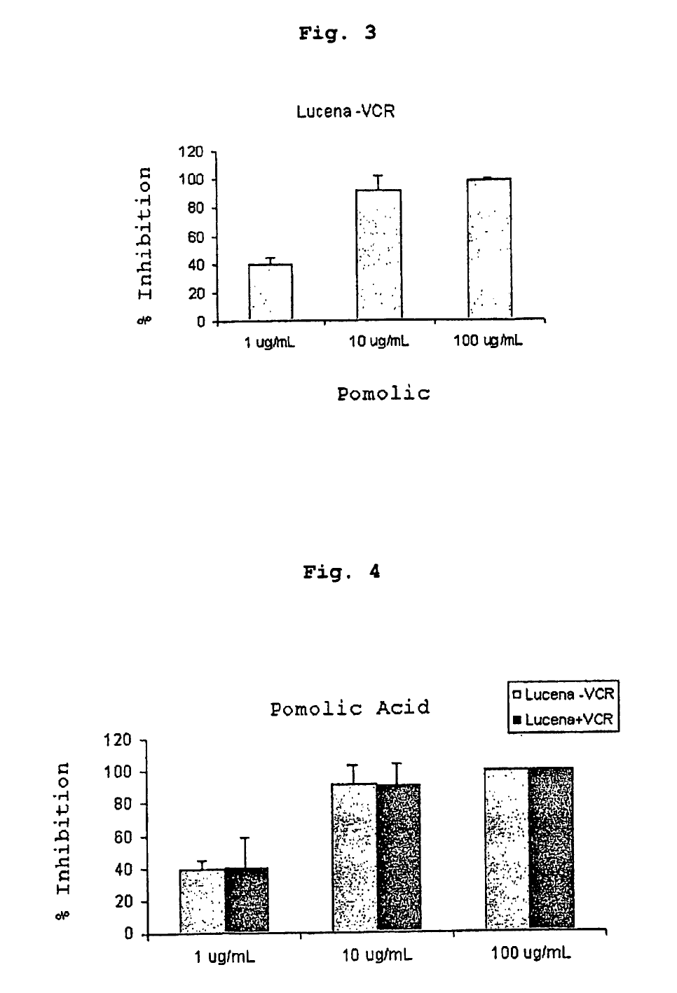

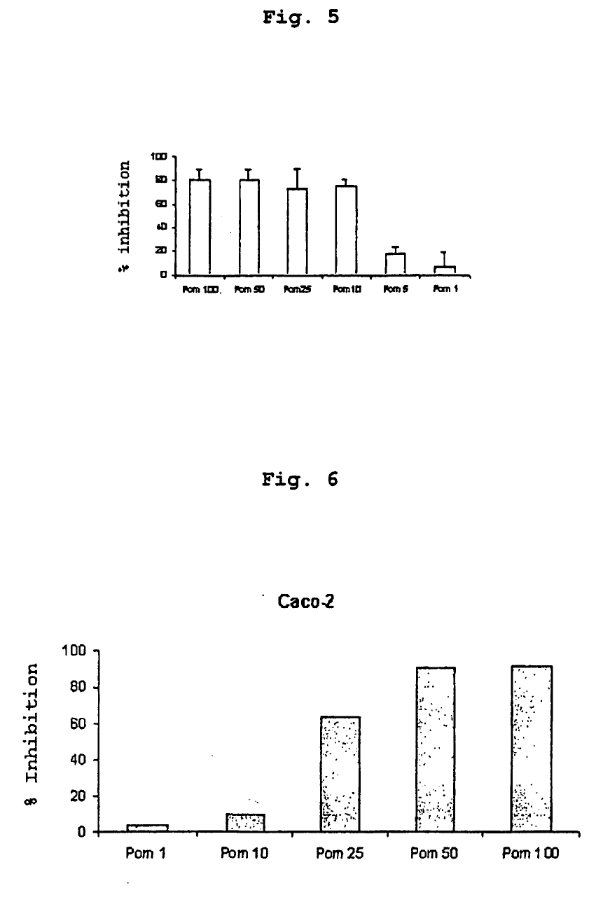

Data of FIG. 2, showing that pomolic acid inhibits Lucena's 1 growth in the presence of vincristine, could suggest a modulatory action over Pgp similar to that of CSA and VP shown on Table 2. However, the fact that the acid kills Lucena 1 in the absence of vincristine, according to FIG. 3, shows that this acid is not a Pgp substrate excluding a possible modulatory role and demonstrating its capacity of acting directly on MDR cells.

Therefore, in addition to anti-tumor activity on sensitive cell lines, the data above puts into evidence the potential of pomolic as a potent anti-MDR agent. The results also highlight a decrease in possible undesirable impacts over the body, as treatment with pomolic acid does not require the use of modulatory agents, which, in general, enhance the side effects of the chemotherapeutic drug.

### Example 6

### Pomolic's Acid Activity over the Tumor Lines with the MDR Phenotype

Pomolic's acid activity was evaluated on cells sensitive to chemotherapeutic agents, such as leukaemic lines (K562 and HL-60), of lung cancer (A549) and throat (HEp-2), and on multidrug resistant of leukaemic origin (Lucena 1), of colon cancer (CaCo-2) and of monkey kidney (MA104). The resistance of Lucena 1 is attributed to the expression of Pgp whereas the resistance of CaCo-2 and MA104 is attributed to Pgp and other MDR genes.

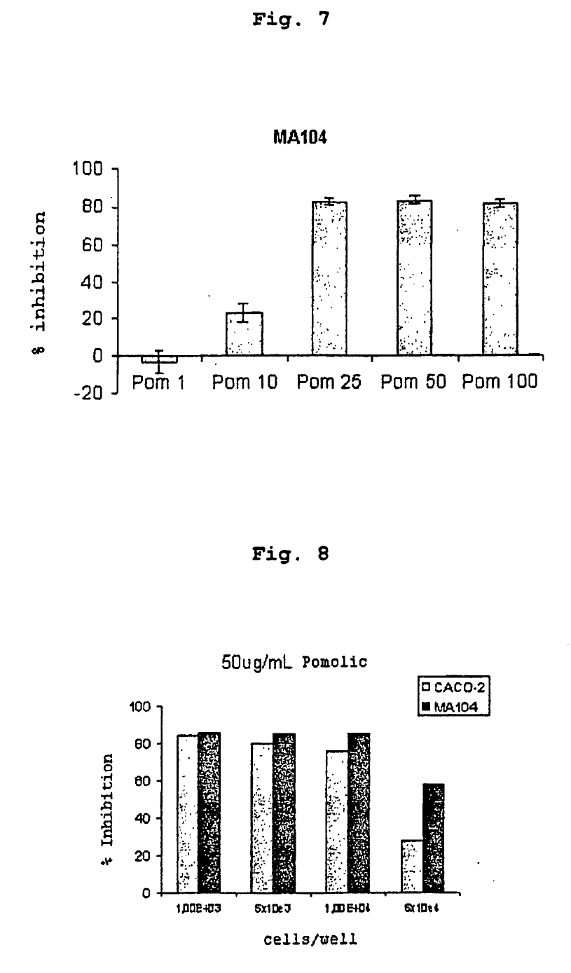

For this 180 μl of the cell suspension (CaCo-2 or MA104) will be delivered to wells in a 96 wells microtiter plate, 2×10⁴ cells/well, and the plate incubated in a CO₂ incubator at 37° C. for 24 h. After this period of time, the cells will be treated with different concentrations of the drug (1, 5, 10, 25, 50 and 100 μg/ml) or of DMSO, incubated for a further 48 h, treated with 20 μl of MTT (5 mg/ml) and processed as described in example 2. The results of FIGS. 6 and 7 express the percentage of inhibition of viability of CaCo2 and MA104 after 48 h treatment with the acid. FIG. 8 presents the effects of 50 μg/ml of pomolic acid over different cells concentrations of these cell lines. These results demonstrate that, not only pomolic acid is capable of directly killing Pgp expressing cells, but it is also of exerting a potent tumoricidal effect on resistant cell lines that express products of other genes capable of inducing MDR.

In conclusion, FIG. 1 shows that pomolic acid inhibits the growth of leukaemic K562 cells (chronic myeloid leukaemia) and FIGS. 2 and 3 show the effect of pomolic acid inhibition on Lucena 1 cells (MDR cell line). The fact that pomolic acid inhibits Lucena's growth in the presence of vincristine, shown in FIG. 2, could suggest a modulatory action over Pgp similar to the one of CSA or VP shown in Table 2. However, the fact that the acid is capable of killing Lucena 1 in the absence of vincristine, as shown in FIG. 3, shows that this acid is not a Pgp substrate, excluding a possible modulatory role and demonstrating its chemotherapeutic activity on MDR cells.

In addition to acting on K562, pomolic acid shows a cytotoxic activity towards other sensitive cell lines such as HL-60 (acute myeloid leukemia), A549 (lung cancer) and HEp-2 (throat cancer), and also resistant ones that express other genes capable of producing MDR through mechanisms other than Pgp such as CaCo-2 (colon cancer) and MA104 (monkey kidney cancer). Pomolic acid's capacity of killing these two resistant cell lines is shown in FIGS. 6, 7 and 8.

For therapeutic applications against multidrug resistant tumors, in accordance with this invention, a therapeutically efficacious amount of pomolic acid, its isomers or derivatives, is administered, orally or systemically. This efficacious amount being comprised preferentially between 0.01 mg/kg and 100 mg/kg body eight.

The chemical composition utilized in this invention contains a sufficient efficacious amount of pomolic acid, its isomers or derivatives and pharmaceutically acceptable vehicles for systemic or oral administration. The pharmaceutically acceptable vehicles consist of a organic solvent, further diluted in a saline solution or another equivalent isotonic solution. The solvent may be dimethylsulfoxide or another organic solvent with acceptable use in humans, diluted in saline solution.

The method to prepare this pharmaceutical composition consists in a first step of solubilization in dimethylsulfoxide or another solvent pharmaceutically acceptable for use in humans and a posterior dilution in a saline solution, in such a way the concentration of dimethylsulfoxide does not exceed 1%, and in this way eliminating the toxic effects of dimethylsulfoxide.

The above description of the present invention was presented with the purpose of illustrating and describing. Furthermore, this description does not intend to limit the invention to the form revealed here, as a consequence, variations and modifications compatible with what is taught above, and the ability or knowledge of relevant technique, are within the scope of the present invention. The modalities described above are meant to explain in a better way the known ways for the use of the invention and to allow the technical personnel in the area to utilize the invention in this or other modalities and with the various modifications necessary for the specific applications or uses of the present invention. It is the intention that the present invention should include all variation and modifications of it, within the scope described in the specification and the claims.

1. An isolated, purified or synthetic compound selected from the group consisting of pomolic acid, isomers of pomolic acid and derivatives thereof, wherein the compound is effective to treat multidrug resistant tumors.

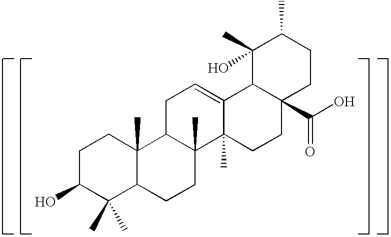

2. A pharmaceutical composition for treating multidrug resistant tumors, said composition comprising an effective amount of at least one compound of claim 1 and at least one pharmaceutically acceptable vehicle.

3. The pharmaceutical composition in accordance with claim 2, wherein the at least one pharmaceutically acceptable vehicle is acceptable for systemic or oral administration.

4. The pharmaceutical composition in accordance with claim 2, wherein the at least one pharmaceutically acceptable vehicle is an organic solvent, further diluted in a saline solution or another equivalent isotonic solution.

5. The pharmaceutical composition, in accordance with claim 4, wherein the organic solvent is dimethylsulfoxide or another organic solvent acceptable to be used in humans, diluted in a saline solution.

6. The pharmaceutical composition, in accordance with claim 2, wherein a concentration of the compound is from 0.1% to 100% in weight/volume.

7. The pharmaceutical composition in accordance with claim 6, wherein the concentration is 10 mg/ml to 1000 mg/ml.

8. A method to prepare a pharmaceutical composition for treating multidrug resistant tumors, said method comprising solubilizing at least one compound selected from the group consisting of pomolic acid, isomers of pomolic acid and derivatives thereof, in a pharmaceutically acceptable solvent for human use, followed by dilution in saline solution, in such a way that a concentration of the pharmaceutically acceptable solvent does not exceed 1%.

9. The method to prepare the pharmaceutical composition in accordance with claim 8, wherein a concentration of the at least one compound is 10 mg/ml to 1000 mg/ml.

10. A method for treatment of multidrug resistance tumors comprising administration of a therapeutically efficacious amount of at least one compound selected from the group consisting of pomolic acid, isomers of pomolic acid and derivatives thereof.

11. The method for treatment in accordance with claim 10, wherein the administration is systemic or oral.

12. The method for treatment in accordance with claim 11, wherein the therapeutically efficacious amount is from 0.01 mg/kg to 100 mg/kg body weight.

13. (canceled)

14. (canceled)
